(19) United States
(12) Patent Application Publication
(10) Pub. No.: US 2023/0015088 A1
(43) Pub. Date: Jan. 19, 2023

(54) CATHETER WITH BICONCAVE SIDE OPENING

(71) Applicant: Bellco S.R.L., Mirandola (IT)

(72) Inventor: Gaspare Marotta, Mirandola (IT)

(21) Appl. No.: 17/783,883

(22) PCT Filed: Dec. 27, 2019
(86) PCT No.: PCT/IT2019/000121

§ 371 Date: Jun. 9, 2022

(51) Int. Cl. A61M 25/00

(52) U.S. Cl. CPC A61M 25/003; A61M 25/007; A61M 25/0032; A61M 2025/0031; A61M 2025/0037

(57) ABSTRACT

A catheter comprising an elongated body (12) defining one or more lumens (40,42), and the elongated body comprises a sidewall defining a side opening (14) in fluid communication with a lumen of the one or more lumens. The side opening has a proximal end (14A), a distal end (14B), a first side (34) extending from the proximal end to the distal end, and a second side (36) opposing the first side and extending from the proximal end to the distal end. When the elongated body is straight, the first and second sides curve towards each other between the proximal and distal ends, e.g., to define a biconcave shape.

### TECHNICAL FIELD

The present disclosure relates to medical catheters.

### BACKGROUND

A medical catheter defining at least one lumen has been proposed for use with various medical procedures. For example, in some cases, a medical catheter may be used to withdraw and introduce fluids to and from body cavities, ducts, blood vessels, and other hollow anatomical structures of a subject. As an example, a catheter may be used in hemodialysis procedures, in which blood is withdrawn from a blood vessel of a patient for treatment and subsequently returned to the blood vessel for circulation.

### SUMMARY

In some respects, this disclosure describes examples catheters that include an elongated body defining one or more side openings having a shape configured to enable an efficient fluid flow in and out of a lumen of the catheter body via the respective side opening, e.g., during a hemodialysis procedure. The side opening shapes described herein may be referred to as biconcave shapes because the side openings are each concave on two sides when the elongated body is straight. In some examples, the biconcave side opening has a proximal end, a distal end, a first side, and a second side opposing the first side, where the first and second sides each extends from the proximal end to the distal end and curve towards each other between the proximal end and the distal end. As an example, the first and second sides may each define a continuous curve that curves towards the other of the first side or the second side in a direction towards a midpoint of the respective first or second side. In addition, the proximal and distal ends of the side opening can be curvilinear in some examples.

In addition to having a biconcave shape, in some examples, the side opening is askew relative to a longitudinal axis of the elongated body when the elongated body is straight, which may further help achieve desired fluid flow properties for one or more medical procedures such as a hemodialysis procedure. The side opening may be askew relative to the elongated body longitudinal axis when, for example, the proximal and distal ends of the side opening are not aligned with each other along an axis parallel to a longitudinal axis of the elongated body when the elongated body is straight and are circumferentially offset in the case of an elongated body that is circular in cross-section. This position of the side opening relative to the longitudinal axis of the elongated body and, in some examples, relative to the direction of fluid flow through a lumen of the elongated body, may further increase the efficiency of fluid flow in and out of a lumen of the catheter body via the side opening.

In some examples, a catheter includes two side openings having biconcave shapes, each side opening being in fluid communication with a respective lumen of the catheter. The side openings may be diametrically opposed from each other and may be axially aligned along a longitudinal axis of the elongated body in some examples or axially offset from each other along the longitudinal axis of the elongated body in other examples.

In some examples, in addition to or instead of the side openings described herein, a catheter includes a catheter tip that defines a tip lumen in fluid communication with only one lumen of the catheter, even in examples in which the catheter includes multiple lumens. In examples in which the catheter tip only has one lumen, the catheter tip may be asymmetrical and/or may have a smaller volume compared to catheter tips of some existing hemodialysis catheters. The smaller volume tip may increase the atraumatic attributes of the catheter and/or may improve navigability of the catheter through vasculature of a patient to a target site.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a perspective view of an example catheter including at least one side opening in fluid communication with a respective lumen.

FIG. 2 is a perspective view of a distal portion of the catheter of FIG. 1.

FIG. 3A is a side elevation view of the distal portion of the catheter of FIG. 1.

FIG. 3B is another side elevation view of the distal portion of the catheter of FIG. 1, and illustrates a side of the catheter different from the side shown in FIG. 3A.

FIG. 3C is another side elevation view of the distal portion of the catheter of FIG. 1, and illustrates a side of the catheter different from the side shown in FIGS. 3A and 3B.

FIG. 4 illustrates an example side opening having a biconcave shape.

FIG. 5 is a schematic cross-sectional view of the distal portion of the catheter of FIG. 1, where the cross-section is taken along line 5-5 in FIG. 3A.

FIG. 6 is a schematic cross-sectional view of the distal portion of the catheter of FIG. 1, where the cross-section is taken along line 6-6 in FIG. 3A.

FIG. 7 is an end view of the distal portion of the catheter of FIG. 1, and illustrates the end of the distal portion when looking at the catheter in a proximal direction.

FIG. 8 is a side elevation view of another example distal portion of a catheter that includes side openings that are not diametrically opposed.

FIG. 9 is a schematic cross-sectional view of the distal portion of the catheter of FIG. 8, where the cross-section is taken in a plane parallel to the image shown in FIG. 8.

FIG. 10 is an exploded view of the catheter of FIG. 1 and an example catheter tip.

FIG. 11 is a side view of the catheter tip of FIG. 10.

FIG. 12 is an end view of the catheter tip of FIG. 10 and illustrates the end of the catheter tip opposite that shown in FIG. 7.

### DETAILED DESCRIPTION

Unless defined otherwise, all technical and scientific terms used generally have the same meaning as commonly understood by a person of ordinary skill in the art.

The articles “a” and “an” are used to refer to one or to more than one (i.e., to at least one) of the grammatical object of the article. For example, “an element” means one element or multiple elements.

In some examples, the term “axis” refers to a fixed reference line.

The term “at least” refers to no less than or at the minimum. For instance, “at least one” could be one or any numbers more than one.

In some examples, such as when used to describe numerical values, “about” or “approximately” refers to a range within the numerical value resulting from manufacturing tolerances and/or within 1%, 5%, or 10% of the numerical value. For example, a length of about 10 mm refers to a length of 10 mm to the extent permitted by manufacturing tolerances, or a length of 10 mm+/−0.1 mm, +1-0.5 mm, or +1-1 mm in various examples.

In some examples, the term “biconcave” refers to being concave on two sides, e.g., concave on two sides of a single opening in the catheter. For example, biconcave shape may refer to a geometric shape that includes at least two concave sides, such as only two concave sides, which may be opposing each other in some examples. The concavity of the sides may be when the shape is projected onto a plane, e.g., in an elevation view.

In some examples, a “continuous curve” refers to an unbroken curve formed along the edge of the opening. In some examples, a continuous curve has a constant radius of curvature. In other examples, a continuous curve as multiple radii of curvature. In addition or instead, in some examples, a continuous curve may not include a sharp point, which can be a point where two separate curves in the opening intersect.

In some examples, the term “distal” refers to a direction toward a patient or a tissue site, and/or away from a clinician.

In some examples, the term “elongated body” refers to a structure (e.g., a tubular member) that is longer than it is wide (e.g., the width being a diameter or another cross-sectional dimension, where the cross-section is taken in a direction orthogonal to a longitudinal axis of the elongated body. In some examples, an elongated body defines at least one lumen (e.g., one lumen, two lumens, three lumens, or more) configured to receive a fluid, a medical device, or both a fluid and a medical device. An elongated body can include, for example, a catheter body that is configured to be at least partially introduced into vasculature of a patient.

In some examples, the term “elongated biconcave shape” refers to a biconcave shape that is longer than it is wide, where, in the case of a side opening, a length of the shape is measured along an axis extending between the proximal and distal ends of the side opening and a width is measured orthogonal to the axis.

In some examples, the term “lumen” refers to a cavity, channel, or bore defined by a structure, e.g., an elongated body or a catheter tip that is separate from and configured to be mechanically connected to an elongated body. In the case of an elongated body, the “lumen” may also be referred to as a “body lumen” and in the case of a catheter tip, the “lumen” may also be referred to as a “tip lumen.” The cavity, channel, or bore can be defined by surfaces of the structure. In some examples, a “body lumen” can particularly refer to a “first body lumen” and a “second body lumen” respectively or single body lumen.

In some examples, the term “proximal” refers to away from a patient or a tissue site, or towards a clinician.

In some examples, a “side opening” of a catheter refers to a through opening, e.g., through a sidewall of a catheter, where the opening permits fluid within the catheter to exit the catheter to an external environment or to allow fluid to enter the catheter through a side wall of the catheter. In some examples, a side opening particularly can refer specifically to a first side opening or a second side opening defined by an elongated body of a catheter, where the side openings define respective apertures, gaps, or other open spaces through which fluid can enter or exit a respective lumen defined by the elongated body.

In some examples, a “sidewall” of a catheter refers to a wall of a catheter body that extends, e.g., from a proximal end to a distal end of the catheter body, and can define an outer surface and/or an inner surface of the catheter.

In examples described herein, a catheter includes a sidewall defining a side opening that is in fluid communication with at least one lumen of the catheter. The configuration of the catheters described herein, including the shape of the side openings, may enable an efficient flow of fluid in and out of a lumen of the catheter via the side opening, e.g., an efficient blood flow exchange during a hemodialysis procedure. As discussed in further detail below, the side openings have biconcave shapes when the elongated body is straight (e.g., not curved or bent so as to skew the shape of side openings relative to when the elongated body is straight). For example, the side openings can each have concave sides on opposite sides of the side opening. When fluid (e.g., blood) flows into a catheter lumen via the biconcave side opening and/or out of the catheter lumen via the biconcave side opening, a relatively low fluid shear stress may be observed at the side opening. A relatively low fluid shear stress may help minimize and even eliminate undesirable effects on fluid flows in the patient body (e.g., breakdown of red blood cells in the blood flow, which is known as homolysis).

In examples of a side opening having a biconcave shape described herein (also referred to herein as a “biconcave side opening”), the concave sides of the side opening may be defined by sides that curve inward towards a side opening axis of the side opening, the side opening axis extending between proximal and distal ends of the opening. In some examples, the side opening axis may also be referred to as a central axis. For example, some example side openings each have a first side and a second side opposing the first side, where the first and second sides each extends from a proximal end of the respective side opening to a distal end of the respective side opening and curve towards each other between the proximal and distal ends. As an example, the first and second sides may each define a continuous curve that curves towards the other of the first side or the second side in a direction towards a midpoint of the respective first or second side, and away from the other of the first side or the second side in a direction away from the midpoint. In some examples, the sides of a biconcave shape that define respective continuous curves (e.g., unbroken curves) have multiple inflection points in conjunction with multiple radii for the curvature variation.

In some examples, a biconcave shape may be symmetrical about the side opening axis of the side opening. In other examples, the biconcave shape may be asymmetrical about the side opening axis of the side opening. For example, the concave sides of side opening may have different radii of curvature from each other.

In some examples, a catheter includes two side openings that are diametrically opposed to each other, e.g., on opposite sides of a central longitudinal axis of the catheter (e.g., of an elongated body of the catheter) from each other. The side openings may each have a biconcave shape. In some examples, the proximal ends, the distal ends, or both the proximal and distal ends of the side openings are aligned along the central longitudinal axis of the catheter. In examples in which both the proximal and distal ends of the side openings are aligned, the side openings may have axial symmetry. In other examples, the both the proximal and distal ends of the side openings are not aligned along the central longitudinal axis of the catheter body. In these examples as well as examples in which only the proximal ends or the distal ends of the side openings are not aligned along the central longitudinal axis of the catheter, the side openings may have axial asymmetry.

The biconcave shape of the side openings described herein generally enables an efficient flow of fluid in and out of a lumen of the catheter via the side opening by providing for a relatively low pressure drop and lower shear stress as fluid flows into or out of the lumen via the side opening. For example, for a fluid flow rate of about 200 milliliters per minute (mL/min) through the catheter lumen having an internal cross-section size of 3.256 square millimeters (mm²)(venous lumen) and 3.281 mm² (arterial lumen) and in/out the side opening with a size of 24.183 mm², a maximum external wall shear stress of the biconcave side opening (at the external surface of the elongated body of the catheter directly adjacent to the side opening) may be about 35 Pascals (Pa) to about 39 Pa and a maximum side wall shear stress (at surface of the side opening extending between the internal and external elongated body surfaces) of about 47 Pa to about 50 Pa. It is believed that these wall shear stresses, as well as a maximum internal wall shear stress (at the internal surface of the elongated body directly adjacent to the side opening) are lower than the shear wall stresses that would be observed with other catheter side opening shapes, such as similarly sized side openings that have straight sides rather than concave sides as described herein.

In addition, due to the shape of the side openings and, in some cases, an axial asymmetry between the side openings and/or the orientation of the central axes of the side openings relative to a longitudinal axis of the elongated body of the catheter, the catheters described herein may help reduce recirculation between two lumens of a catheter during a hemodialysis procedure. Recirculation can occur when purified blood exiting one lumen of a catheter, referred to as a venous lumen in some examples, is withdrawn directly into another lumen of the catheter, referred to as an arterial lumen in some examples, such that purified blood is returned to a dialysis device (referred to herein as a dialyzer in some examples). Recirculation can increase the time required to complete the hemodialysis procedure because blood that has already been run through a dialyzer may be run through the dialyzer again, which may be unnecessary to achieve the desired results of the hemodialysis procedure. In examples described herein, due at least in part to the configuration of the side openings via which blood is introduced into and exits the catheter lumens, the blood flow stream into a side opening in fluid communication with an arterial lumen of the catheter and the blood flow stream exiting a side opening in fluid communication with a venous lumen are separated such that the degree of fluid recirculation is minimized. For example, the diametrically opposed positioning of the side openings along a distal portion of catheter, and an elongated biconcave shape of side openings enables the spacing between the fluid stream exiting the venous lumen and the fluid stream entering the arterial lumen to be increased, which can minimize the degree of recirculation of purified blood between the venous lumen and the arterial lumen of the catheter.

In some examples, in addition to or instead of the side openings described herein, a catheter includes an atraumatic distal catheter tip, which defines a tip lumen in fluid communication with only one lumen of the catheter. The tip configuration may have a smaller volume (e.g., may be relatively short and/or narrow) compared to some existing hemodialysis catheters that includes catheter tips define multiple lumens in fluid communication with respective lumens of the catheter, which may increase its atraumatic attributes and may improve navigability of the catheter through vasculature of a patient to a target site. In addition, in some examples, the catheter tip shape is configured to improve blood outflow via the lumen in fluid communication with the lumen of the catheter tip compared to catheters that have longer and/or wider tips.

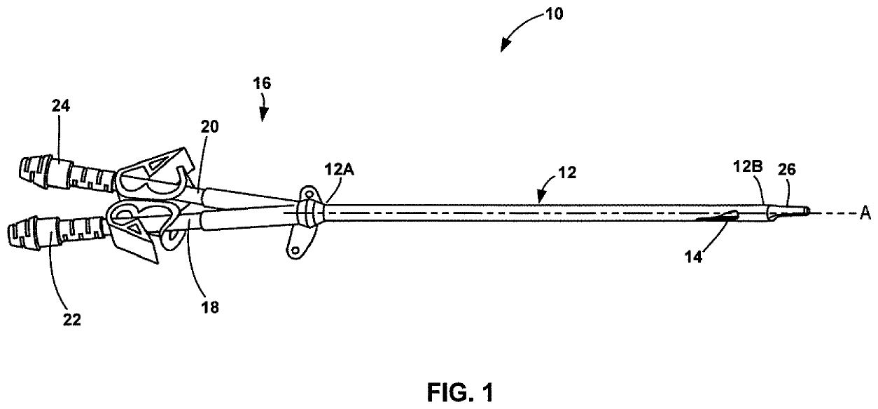

FIG. 1 is a perspective view of an example catheter 10, which includes an elongated body 12 defining at least one side opening 14 in fluid communication with a lumen (not shown in FIG. 1) defined by elongated body 12. Catheter 10 defines a longitudinal axis A, which is parallel to a longitudinal axis of elongated body 12, and, therefore, may be interchangeably be referred to herein as a longitudinal axis A of elongated body 12. In some examples, longitudinal axis A is also a central longitudinal axis of catheter 10 and elongated body 12. Catheter 10 further comprise a hub 16, which is positioned at a proximal end 12A of elongated body 12, and catheter tip 26.

Hub 16 is at a proximal portion of catheter 10 and is configured to fluidically connect the one or more lumens defined by catheter 10 to another device, such as a hemodialysis device (e.g., a dialyzer). Hub 16 can be attached to the elongated body 12 in any suitable manner, such as by injection molding a body of hub 16 onto elongated body 12 at the proximal end 12A. In the example shown in FIG. 1, hub 16 includes extension tubes 18, 20 and adapters 22, 24. Each extension tube 18, 20 is in fluid communication with a respective lumen of elongated body 12 via an associated fluid passage extending through the hub 16. A clinician may fluidically couple a device, such as a hemodialysis device, to elongated body 12 via extension tubes 18, 20. Adapters 22, 24 can be positioned on distal ends of the extension tubes 18, 20, respectively, opposite to the ends of the extension tubes secured to hub 16 to facilitate the connection of a device to extension tubes 18, 20. For example, adapter 22, 24 can be or otherwise include Luer-lock adapters.

Elongated body 12 may also be referred to as a catheter body in some examples. Elongated body 12 can have any suitable shape (e.g., tubular), and may be made of any suitable material that provides desired properties, such as, but not limited to, a polymer (e.g., polyurethane and/or silicone), and can have any suitable dimensions, depending on the type of the catheter 10. The elongated body 12 is flexible and may be formed by any suitable process, such as, but not limited to, injection molding or extrusion. In some examples, elongated body 12 is substantially straight (e.g., straight or nearly straight to the extent permitted by manufacturing tolerances) in its “at rest” state in the absence of external forces applied to elongated body 12 to change its shape. In other examples, elongated body 12 may have a preformed bend to facilitate conforming to an internal body cavity or vessel in which elongated body 12 is to be positioned. Although an elongated body 12 having a circular cross-section is primarily referred to herein, the cross-section being taken in a direction orthogonal to longitudinal axis A, in other examples, elongated body 12 may define any suitable shape in cross-section, such as, but not limited to, elliptical, square, triangular, and the like.

Elongated body 12 can have any suitable dimensions. For example, in some cases, elongated body 12 has an outer diameter from about 4 French to about 12 French (where 1 French is ⅓ mm), such as about 5 French.

Elongated body 12 defines one or more lumens configured to receive a fluid. The number of lumens may depend on the function of catheter 10. For example, elongated body 12 may define two lumens or three lumens in some examples in which catheter 10 is intended to be used a hemodialysis catheter. In examples in which elongated body 12 defines two or more lumens, catheter 10 can be employed for simultaneous withdrawal and introduction or return of fluid (e.g., blood) from a patient.

For example, catheter 10 may be configured to be used to perform hemodialysis, hemodiafiltration, ultrafiltration, or like on a patient in which toxins are removed from a body of a patient. At least a distal portion of catheter 10 can be inserted into a body of a patient and a proximal portion of catheter 10 may be connected to a dialysis unit (e.g., a dialyzer), such as via extension tubes 18, 20, and adapters 22, 24. Blood can be withdrawn from the patient through one lumen (referred to in some examples as an arterial lumen) defined by elongated body 12 and supplied to the dialysis unit, which dialyzes, or cleans, the blood to remove waste and excess water. The dialyzed blood is returned to the patient through a different lumen (referred to in some examples as a venous lumen) defined by elongated body 12.

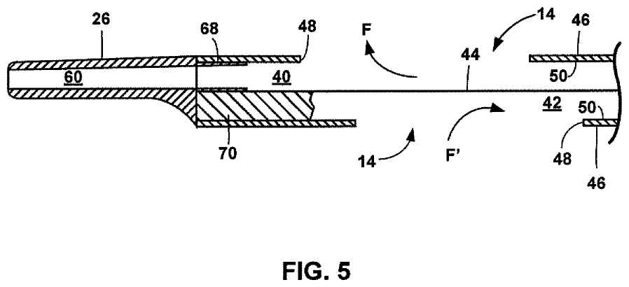

Elongated body 12 includes a sidewall 28 defining one or more side openings 14, through which blood or another fluid can enter or exit a lumen of elongated body 12. In the example shown in FIG. 1 (and as better shown in FIG. 5, described below), elongated body 12 defines two lumens 40, 42 (FIG. 5) and two side openings 14, which are each in fluid communication with a respective lumens 40 and 42. In other examples, two or more side openings may be in fluid communication with the same lumen of elongated body 12, or one of the lumens of elongated body 12 may only be open at a distal opening at a distal-most end 12B of elongated body 12 and may not be open to an exterior of elongated body 12 via a side opening 14 defined by sidewall 28.

Elongated body 12 can define any suitable number of side openings 14. In the example shown in FIG. 1, elongated body 12 defines two side openings 14. In other examples, elongated body 12 can include only one side opening or three or more side openings. Although a two-lumen catheter 10 is primarily referred to herein, in other examples, elongated body 12 can define only one lumen or more than two lumens (e.g., three lumens), and at least one of the lumens can be in fluid communication with an exterior of catheter 10 via a respective side opening, which can have at least one biconcave shape in accordance with examples described herein or another shape.

In the example shown in FIG. 1, side openings 14 are positioned on different sides of elongated body 12, such that the two side openings 14 are not circumferentially aligned (only one opening 14 shown). For example, side openings 14 may be diametrically opposed from each other, and, as a result, are positioned on opposite sides of longitudinal axis A from each other. Side openings 14 can have any suitable alignment with each other along longitudinal axis A, referred to herein as an axial alignment. For example, the proximal ends 14A of side openings 14, the distal ends 14B of side openings 14, or both the proximal and distal ends 14A, MB of side openings 14 can be aligned along longitudinal axis A (referred to herein as being “axially aligned” in some examples). In some examples, the proximal ends 14A and the distal ends 14B of the side openings 14 are longitudinally aligned with each other along longitudinal axis A, such that the two side openings 14 are axially aligned and have axial symmetry. In other examples, the proximal ends 14A and/or the distal ends MB of the side openings 14 are not longitudinally aligned with each other along longitudinal axis A, such that side openings 14 have axial asymmetry. In examples in which both the proximal and distal ends 14A, 14B of side openings 14 are not longitudinally aligned with each other, the side openings 14 may be referred to as being longitudinally offset from each other.

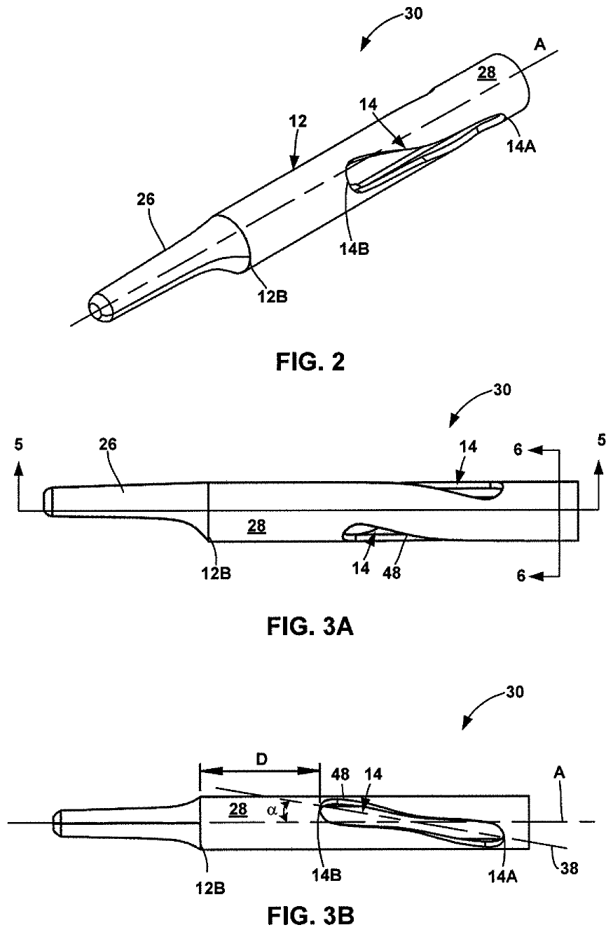

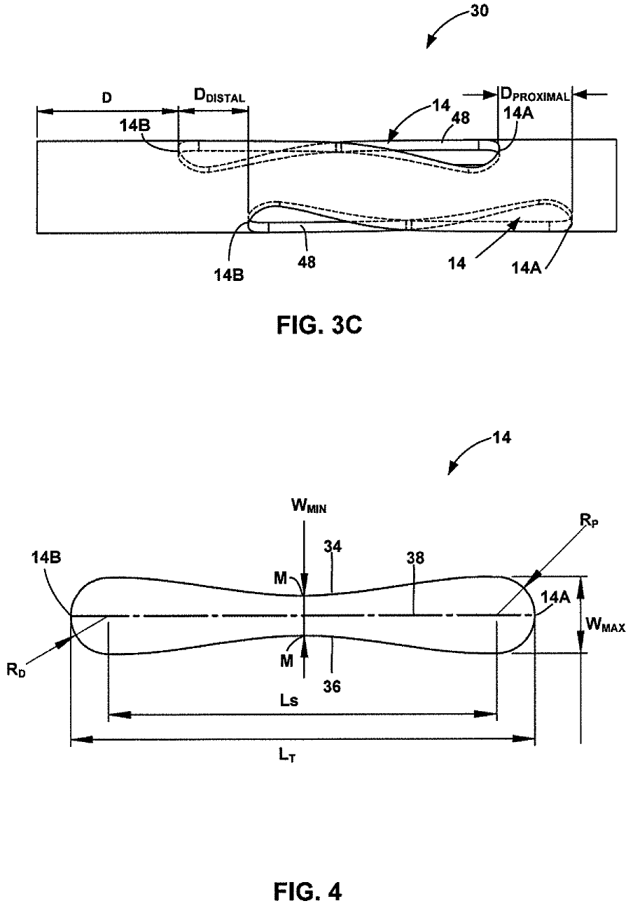

As shown in FIGS. 2-4, side openings 14 are each concave on two sides and, therefore, each have a biconcave shape. FIG. 2 is a perspective view of a distal portion of catheter 10 and illustrates a distal portion 30 of elongated body 12 and catheter tip 26. The distal portion of catheter 10 includes a distal portion of elongated body 12, and catheter tip 26, which defines the distal-most end of catheter 10. FIGS. 3A-3C are side elevation views of distal portion 30 of elongated body 12 shown in FIG. 2, and illustrate elongated body 12 in different rotational positions (the axis of rotation being parallel to longitudinal axis A). FIG. 4 illustrates a projection of side opening 14 in a plane and illustrates an example biconcave shape. Side openings 14 of the example catheter 10 shown in FIGS. 1-6 have the same shape and size (e.g., lengths and widths). Thus, the description of one side opening 14 applies to the other side opening 14. In other examples, the side openings 14 may have different shapes and/or different sizes (e.g., different maximum widths and/or different maximum lengths).

As shown in FIGS. 2-4, side opening 14 has a proximal end 14A, a distal end 14B, a first side 34, and a second side 36 opposing the first side 34. Sides 34,36 each extends between proximal and distal ends 14A, 14B. Proximal end 14A, distal end 14B, and sides 34,36 define the outer perimeter of side opening 14 in the longitudinal direction of the catheter, which itself is physically defined by sidewall 28 of elongated body 12. Proximal end 14A can be an end-most part of side opening 14 and distal end 14B can be an end-most part of side opening 14 opposite the proximal end 14A. Side opening axis 38 (FIGS. 3B and 4) extends between proximal end 14A and distal end 14B, e.g., intersects both proximal and distal ends 14A, 14B. In examples in which side opening axis 38 is parallel to longitudinal axis A of elongated body 12, such that angle α shown in FIG. 3 is 0°, proximal end 14A is the portion (e.g., the point) of side opening 14 closest to proximal end 12A of elongated body 12 and distal end 14B is the portion (e.g., the point) of side opening 14 closest to distal end 12B of elongated boy 12. That is, in examples in which side opening axis 38 is parallel to longitudinal axis A of elongated body 12, proximal end 14A is the proximal-most part of side opening 14 and distal end 14B is the distal-most part of side opening 14.

In examples in which side opening 14 is askew relative to longitudinal axis A of elongated body 12 (as shown in the examples of FIGS. 2-3C), such that side opening axis 38 is not parallel to longitudinal axis A but is instead transverse to longitudinal axis A and angle α shown in FIG. 3B is, for example, greater than 0° with a maximum rotation equal to 15°, and wherein the proximal end 14A of side opening 14 is not the portion (e.g., the point) of side opening 14 closest to proximal end 12A of elongated body 12 and distal end 14B is not the portion (e.g., the point) of side opening 14 closest to distal end 12B of elongated boy 12. Further, in these examples, proximal end 14A is not aligned with distal end 14B along an axis parallel to longitudinal axis A. In some examples, side opening 14 is oriented relative to longitudinal axis A such that side opening axis 38 is at an angle α of about 5° to about 15° relative to longitudinal axis A, such as about 11° to about 15, or about 11.2°. Angle α may be selected such that side opening 14 is only open to one lumen 40 or 42, and does not cross over to be open to the other lumen of catheter 10.

Side openings 14 may each be positioned any suitable distance from distal end 128 of elongated body 12. In some examples, distal end 14B of side opening is a distance D of about 1 mm to about 15 mm from distal end 12B of elongated body 12, such as about 3 mm to about 10 mm, or about 6 mm.

As discussed above, in some examples, distal ends 14B and/or proximal ends 14A of the two side openings 14 shown in the example of FIGS. 1-3C may be aligned. In other examples, side openings 14 are axially displaced from each other, e.g., because distal ends 14B and/or proximal ends 14A of the two side openings 14 shown in the example of FIGS. 1-3C are not aligned. Axial displacement between at last partially diametrically opposed side openings 14 may enable fluid recirculation between lumens of catheter 10 to minimized, as discussed in further detail below.

As shown in FIG. 3C, in some examples, distal end 14B of one side opening 14 is axially displaced from distal end 14B of the other side opening (along longitudinal axis A) by a distance D_{DISTAL} of about 1 mm to about 10 mm, such as about 2 mm to about 5 mm, or about 3 mm. In addition or instead, in some examples, proximal end 14A of one side opening 14 is axially displaced from proximal end 14A of the other side opening (along longitudinal axis A) by a distance D_{PROXIMAL} of about 1 mm to about 10 mm, such as about 2 mm to about 5 mm, or about 3 mm. The distances D_{DISTAL} and D_{PROXIMAL} can be the same in examples and different in other examples. In examples in which one or both distances D_{DISTAL} and D_{PROXIMAL} is greater than zero and side openings 14 are on opposite sides of longitudinal axis A, side openings 14 may be considered partially diametrically opposed.

Further, in some examples, one or both of distances D_{DISTAL} and D_{PROXIMAL} is substantially zero (e.g., zero or nearly zero to the extent permitted by manufacturing tolerances). In examples in which distances D_{DISTAL} and D_{PROXIMAL} are both substantially zero and side openings 14 are on opposite sides of longitudinal axis A, side openings 14 may be considered fully diametrically opposed.

As shown in FIGS. 2-4, proximal and distal ends 14A, 14B are curvilinear in some examples, which may also be referred to as being “rounded” ends. In the example shown in FIGS. 2-4, proximal end 14A has a radius of curvature R_{P} and distal end 14B has a radius of curvature R_{D}. In some examples, radii of curvature R_{P} and R_{D} are substantially equal to each other (e.g., equal to each other to the extent permitted by manufacturing tolerances). In other examples, radii of curvature R_{P} and R_{D} are different from each other. In some examples, proximal end radius of curvature R_{P} and distal end radius of curvature R_{D} are each about 1 millimeters (mm) to about 1.5 mm, such as about 1 mm to about 1.25 mm or about 1.15 mm. Other radii of curvature can be used for proximal and distal ends 14A, 14B in other examples.

Sides 34, 36 of side opening 14 curve towards side opening axis 38 to define respective concave sides of biconcave side opening 14. That is, sides 34, 36 each curve towards side opening axis 38 in a direction away from both proximal end 14A and distal end 14B of side opening 14. Sides 34, 36 may each define continuous (unbroken) curves, e.g., as shown in FIGS. 2-4, but may have differing curves including points of inflection. In some examples, sides 34, 36 curve towards each other in a direction towards a midpoint M of side opening 14 and subsequently away from each other in a direction away from midpoint M. For example, sides 34, 36 may curve towards each other from proximal end 14A towards midpoint M and then away from each other from midpoint M to distal end 14B. Midpoint M can be, for example, a longitudinal center of side opening 14. In these examples, sides 34, 36 are closest to each other at midpoint M. In other examples, sides 34, 36 may be closest to each other at a point along side opening axis 38 other than the midpoint M.

In some examples, a radius of curvature defined by sides 34, 36 is about the same, such that sides 34, 36 are symmetric about side opening axis 38. In other examples, sides 34, 36 have different radii of curvature, such that sides 34, 36 are asymmetric about side opening axis 38. In some examples, side opening 14 is symmetric about an axis transverse to side opening axis 38, e.g., extending transverse to side opening axis 38 and through midpoints M. In other examples, such as examples in which proximal and distal ends have different radii of curvature R_{P} and R_{D}, respectively, side opening 14 is asymmetric about the axis transverse to side opening axis 38.

Side opening 14 has any suitable total length L_{T} (also referred to herein as a maximum length) measured along side opening axis 38 from proximal end 14A to distal end 14B. The total length L_{T}, as well as other lengths of side opening 14 described herein, may be selected based on one or more factors, such as, but not limited to, the desired fluid flow properties of catheter 10, the size of lumens 40, 42 (FIG. 5) defined by elongated body 12, and the overall length of elongated body 12 (measured from proximal end 12A to distal end 12B along longitudinal axis A). A longer length L_{T} may contribute to reducing a pressure drop as fluid exits a lumen 40 or 42 of catheter via the respective side opening having the longer length L_{T}. In some examples, length L_{T} of side opening 14 is in a range of about 5 mm to about 25 mm, such as about 10 mm to about 20 mm, or about 14 mm.

Sides 34, 38 of side opening 14 have any suitable length L_{S}, which is less than length L_{T} of side opening 14 and is measured along side opening axis 38 between proximal end distal ends 14A, 14B. For example, in the example shown in FIG. 4, the length of each side 34, 38 is the total length L_{T} minus the radius of curvature R_{P} of proximal end 14A and minus the radius of curvature R_{D} of distal end 14B. That is, length L_{S} of each side 34, 38 may be measured along side opening axis 38 from a center of curvature of radius of curvature R_{P} to a center of curvature of radius of curvature R_{D}. In some examples, length L_{S} of sides 34, 38 is in a range of about 2 mm to about 23 mm, such as about 8 mm to about 16 mm, or about 12 mm.

Side opening 14 has any suitable maximum width W_{MAX}, which is measured along an axis orthogonal to side opening axis 38. In some examples, side opening 14 is widest, and, therefore, maximum width W_{MAX} occurs, at the distal-most part of proximal end 14A and at the proximal-most part of distal end 14B, e.g., where the ends 14A, 14B begin to curve. In some examples, maximum width W_{MAX} is about 1 mm to about 3 mm, such as about 2 mm to about 2.5 mm, or about 2.3 mm.

A width of side opening 14 varies along its length. Side opening 14 defines a minimum width W_{MIN}, which is measured along an axis orthogonal to side opening axis 38. As discussed above, sides 34, 36 curve towards side opening axis 38, and, thus, in some examples, side opening 14 is the narrowest and, therefore, has its minimum width W_{MIN} where sides 34, 36 are closest to each other. In some examples, such as examples in which sides 34, 36 are closest to each other at their respective midpoints (along side opening axis 38) and in which proximal end distal ends 14A, 14B have substantially the same radii of curvature (e.g., the same to the extend permitted by manufacturing tolerances), minimum width W_{MIN} of side opening occurs at a midpoint of side opening 38 along side opening axis 38. In some examples, minimum width W_{MIN} is about 0.2 mm to about 2.2 mm, such as about 1 mm to about 1.5 mm, or about 1.2 mm. Side opening 14 can have other dimensions in other examples. In some examples, the size of side opening 14 can be scaled up or down for other catheter sizes in proportion to the diameter of the catheter. For example, if a side opening has a particular width and length for a 12 French catheter provided above are for a 12 French catheter, then the width and length can be scaled by a ratio of 0.3325 for a 4 French catheter.

The biconcave shape of side openings 14, may enable an efficient blood flow exchange during a hemodialysis procedure. For example, the shape of side openings 14 is configured to provide a relatively low blood shear stress during the inflow of blood into a lumen of elongated body 12 via the respective side opening 14 and/or during the outflow of blood from the lumen via the respective side opening 14. The shape of the side openings also provides for a relatively low pressure drop.

In addition, due to the shape of side openings 14 and the relative position of the side openings 14, catheter 10 is configured to reduce recirculation between the arterial and venous lumens defined by elongated body 12. As discussed above, recirculation can occur during a hemodialysis procedure when purified blood exiting the venous lumen of the catheter is withdrawn directly into the arterial lumen such that purified blood is returned to the dialyzer. As such, recirculation can increase the time required to complete the hemodialysis procedure. Because of the asymmetrical configuration of side openings 14 in the example shown in FIGS. 2-3, the blood flow stream into a side opening in fluid communication with an arterial lumen of the catheter and the blood flow stream exiting a side opening in fluid communication with a venous lumen are separated such that the degree of fluid recirculation is minimized. For example, the at least partially diametrically opposed positioning of the side openings 14, the elongated shape of side openings 14, and the orientation of side opening axis 38 transverse to longitudinal axis A may enable the spacing between the fluid stream exiting the venous lumen and the fluid stream entering the arterial lumen to be increased, which minimizes the degree of recirculation of purified blood between the venous lumen and the arterial lumen of the catheter.

FIG. 5 is a schematic cross-sectional view of distal portion 30 of catheter 10, where the cross-section is taken along line 5-5 in FIG. 3A along central longitudinal axis A of catheter 10. As shown in FIG. 5, elongated body 12 defines at least one lumen, which is shown in FIG. 5 as two lumens 40, 42, which are each in fluid communication with at least one side opening 14 to enable fluid from the at least one lumen to enter and exit elongated body 12. In some examples in which elongated body 12 includes two or more lumens, elongated body 12 may define or otherwise include a septum 44 that extends along a length of elongated body 12, and defines at least a part of the two or more lumens. In the example shown in FIG. 5, septum 44, together with sidewall 28, defines lumens 40, 42. In other examples, as discussed above, catheter 10 can have a single lumen or more than two lumens. For example, elongated body 12 may define a third lumen for receiving a guidewire or the like.

Lumens each 40, 42 define fluid conduits of catheter 10. In some examples, lumens 40, 42 each extend from proximal end 12A of elongated body 12 through elongated body 12 to distal end 12B and terminate at distal end 12B. Lumens 40, 42 may be fluidically accessed at the proximal portion of catheter 10 via hub 16. For example, when hub 16 is properly connected to elongated body 12, lumen 40 may be in fluid communication with extension tube 20 and adapter 24, and lumen 42 may be in fluid communication with extension tube 18 and adapter 22.

Side openings 14 are configured to enable fluid streams F, F′ to travel between an environment external to elongated body 12, such as vasculature or other hollow anatomical structure of a patient, and the internal lumens 40, 42 of elongated body 12. Side openings 14 may have contoured edges formed, for example, by laser cutting, molding with elongated body 12, and/or otherwise smoothed to minimize blood flow disruption (or hemolysis) and thrombus formation. The contoured edges can be, for example, at a junction between outer surface 46 of elongated body 12 and a side surface 48 defining side opening 14, where the side surface 48 extends between outer surface 46 and inner surface 50 of elongated body 12. Inner surface 50 can define, for example, at least part of lumens 40, 42.

Because of the configuration of elongated body 12, either lumen 40, 42 may serve as the arterial lumen or the venous lumen during a hemodialysis procedure. Thus, catheter 10 may be a reversable hemodialysis catheter. Fluid may outflow in one axial direction through one lumen 40 or 42 and flow in the opposite axial direction through the other lumen 42 or 40. Blood flow stream F′ entering into side opening 14 in fluid communication with lumen 42 (and not in fluid communication with lumen 40) and the blood flow stream F exiting side opening 14 in fluid communication with the other lumen 40 (and not in fluid communication with lumen 42) are separated such that the degree of fluid recirculation is minimized. That is, outer surface 46 of elongated body 12 provides spacing between the two side openings 14 that substantially minimizes the fluid stream F exiting lumen 40 acting as an arterial from migrating toward the fluid stream F′ entering lumen 42 acting as a venous lumen.

Lumens 40, 42 can have any suitable cross-sectional shape and size that provides the desired fluid flow characteristics through the lumens. In some examples, lumens 40, 42 may include oblong, kidney-shaped, and/or D-shaped cross-sectional configurations.

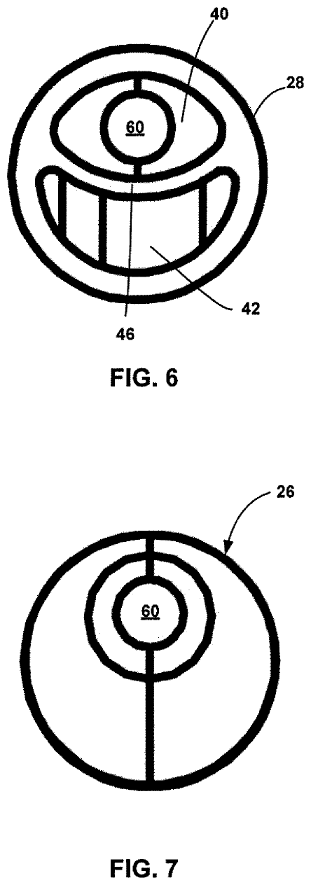

FIG. 6 is a schematic cross-sectional view of elongated body 12, where the cross-section is taken in a direction orthogonal to longitudinal axis A along line 6-6 in FIG. 3A. In the example shown in FIG. 6, lumen 42 has a kidney-shape in cross-section and lumen 40 has a shape resulting from the occupation of the kidney-shape in a circular cross-section. Also shown in FIG. 6 is lumen 60 defined by catheter tip 26, which is described in further detail below with reference to FIGS. 7 and 10-12. In other examples, lumens 40, 42 may each have a different shape in cross-section. For example, one or both lumens 40 42 may have a “D” shape in cross-section (e.g., defined by respective longitudinal halves of elongated body 12), may have a “C” shape in cross-section, may have a kidney shape in cross-section, may have a circular shape in cross-section, may have a polygon shape in cross-section, or the like.

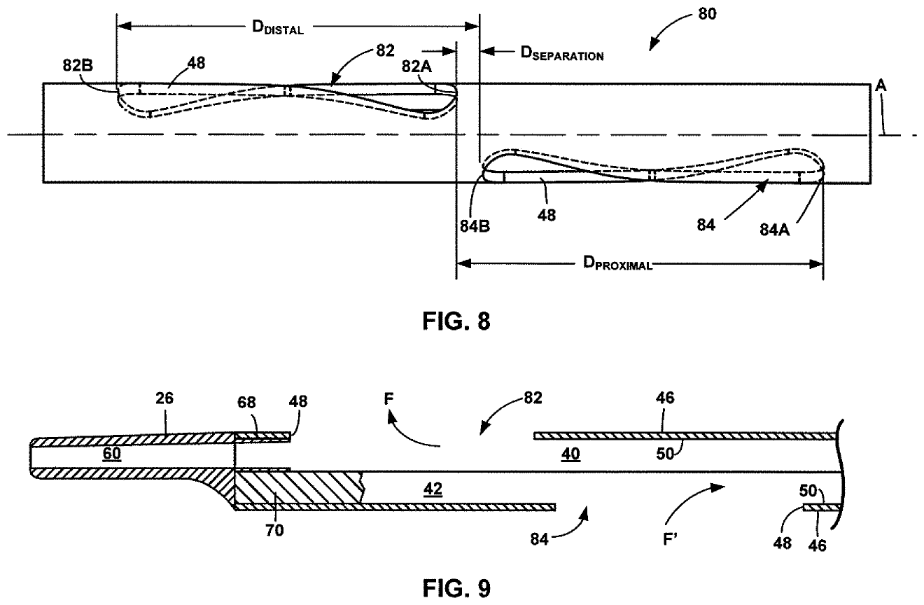

Although FIGS. 2-3C and 5 illustrate an example catheter 10 that includes diametrically opposed side openings 14, in other examples, side openings of a catheter are not diametrically opposed (e.g., coextensive along longitudinal axis A), and are instead entirely longitudinally displaced from each other. FIG. 8 is an example side elevation view of another example distal portion 80 of an elongated body of a catheter, which is an example of elongated body 12 and catheter 10 shown in FIG. 1. Thus, the elongated body shown in FIG. 8 will be referred to as elongated body 12. FIG. 9 is a schematic cross-sectional view of the distal portion of the catheter of FIG. 8, where the cross-section is taken in a plane parallel to the image shown in FIG. 8. FIG. 9 also illustrates catheter tip 26, e.g., as described with reference to a similar cross-sectional view of FIG. 5.

In the example shown in FIGS. 8 and 9, elongated body 12 defines side openings 82, 84, which are identical to side openings 14 except for the position of the side openings on opposite sides of longitudinal axis A relative to each other. Thus, side openings 82, 84 shown in FIG. 8 are identical in shape to side openings 14 of FIGS. 1-6, but instead of being diametrically opposed, side openings 82, 84 are entirely longitudinally displaced from each other. That is, in contrast to side openings 14 of FIGS. 1-6, side openings 82, 84 are not coextensive along longitudinal axis A of elongated body 12. Proximal end 82A of side opening 82 is separated from distal end 84B of side opening 84 by distance D_{SEPARATION}, which is measured along longitudinal axis A. In some examples, distance D_{SEPARATION} is about 1 mm to about 25 mm, such as about 5 mm to about 10 mm, or about 2 mm.

The axial separation distance D_{SEPARATION} between side openings 82, 84 contributes to the axial displacement (distance D_{DISTAL}) between distal end 82B of side opening 82 and distal end 84B of side opening 84 (along longitudinal axis A) and the axial displacement (distance D_{PROXIMAL}) between proximal end 82A of side opening 82 and proximal end 84A of side opening 84. In some examples of distal portion 80, distance D_{DISTAL} is about 15 mm to about 39 mm, such as about 15 mm to about 20 mm, or about 16 mm, and distance D_{PROXIMAL} is about 15 mm to about 39 mm, such as about 15 mm to about 20 mm, or about 16 mm.

Side openings 82, 84 that are entirely longitudinally displaced from each other, as shown in FIGS. 8 and 9, and do not overlap along longitudinal axis A, enables the spacing between the fluid stream F entering lumen 42 and the fluid stream F′ exiting lumen 40 to be increased, which can minimize the degree of recirculation of purified blood between the venous lumen and the arterial lumen of catheter 10 during hemodialysis. Thus, the non-diametrically opposed positioning of the side openings 82, 84 and the biconcave of side openings 82, 84 may help separate fluid stream F exiting a venous lumen and fluid stream F′ entering an arterial lumen, which may contribute to better treatment outcomes or at least shorten the duration of a dialysis session.

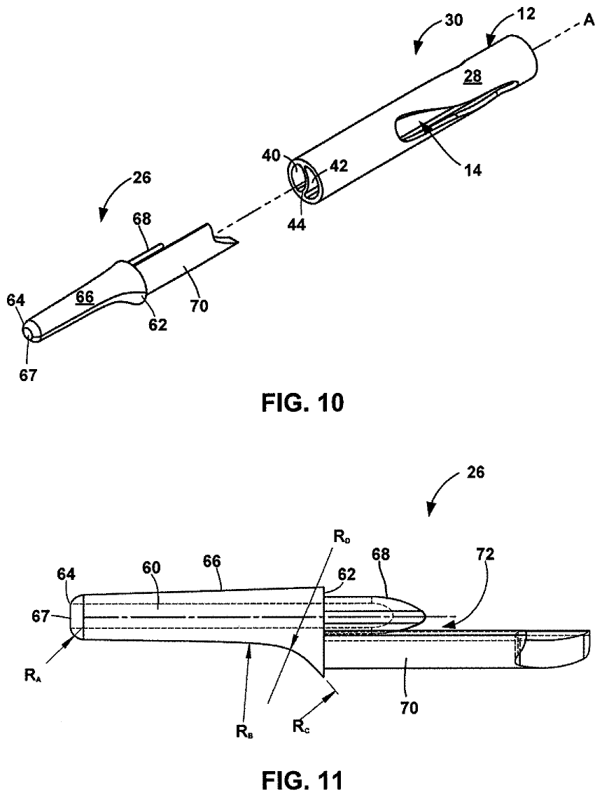

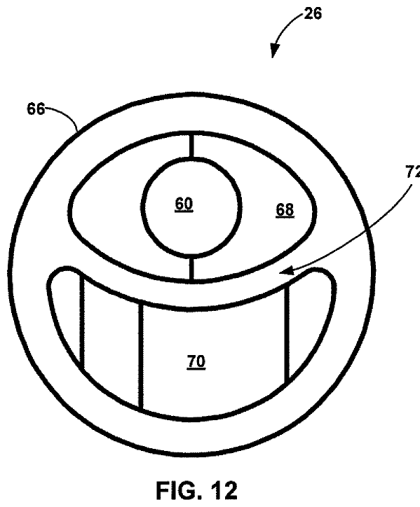

As shown in FIGS. 1-3B, as well as FIGS. 6, 7, and 9-12, in some examples, catheter 10 includes catheter tip 26, which is positioned at distal end 12B of elongated body 12. FIG. 7 is an end view of catheter tip 26, looking at distal end 64 of catheter tip 26 in a direction towards a proximal end 62 of catheter tip 26. FIG. 10 is an exploded perspective view of distal portion 30 of catheter 10 and illustrates catheter tip 26 aligned with, but not mechanically connected to elongated body 12. FIG. 11 is a side elevation view of catheter tip 26.

Catheter tip 26 defines a distal-most portion of catheter 10 and, therefore, may be configured to help aid insertion of catheter 10 into a patient. For example, an outer surface 66 of catheter tip 26 is tapered distally to aid insertion of the catheter 10 into vasculature or another hollow anatomical structure of a patient. While distal end 64 of catheter tip 26 is shown as having a rounded, blunt profile, other shapes and profiles of distal end 64 can also be used. In some examples, distal end 64 has a relatively atraumatic profile, e.g., as defined by the distal taper and rounded distal end 64.

Outer surface 66 of catheter tip 26 and outer surface 46 of elongated boy 12 may have similar configurations, such that when catheter tip 26 and elongated body 12 are properly assembled, the junction between catheter tip 26 and elongated body 12 is relatively smooth (e.g., have a substantially uniform outer perimeter, such as substantially the same outer diameters). A relatively smooth transition between catheter tip 26 and elongated body 12 may help reduce the possibility that thrombi will form from the flow of blood across the junction and may also help provide a smoother surface to engage with patient tissue.

Catheter tip 26 may be fabricated from material suitable for medical application, including, for example, polymers, silicone, and/or polyurethane. In addition, catheter tip 26 fabricated from the same material or a different material than elongated body 12. In some examples, catheter tip 26 is formed separately from elongated body 12 and is secured to a distal portion of elongated body 12, such as distal end 12B of elongated body 12. In other examples, the catheter tip 26 is integrally or monolithically formed with elongated body 12. At least a distal portion of elongated body 12 and a proximal portion of catheter tip 26 have substantially similar outer dimensions to provide a smooth transition between elongated body 12 and the catheter tip 26.

Catheter tip 26 defines lumen 60, which extends from proximal end 62 of catheter tip 26 to distal end 64. As shown in FIG. 11, lumen 60 terminates a distal opening 67 at the distal-most end 64 of catheter tip 26. Distal opening 67 may be a distal-most opening of catheter 10. In some examples, when elongated body 12 and catheter tip 26 are properly assembled, lumen 60 of catheter tip 26 is in fluid communication with and is aligned with only one lumen 40, 42 of elongated body 12. In the example shown in the figures, lumen 60 of catheter tip 26 is in fluid communication with lumen 40 of elongated body 12 when elongated body 12 and catheter tip 26 are properly assembled. In these examples, lumen 42 of elongated body 12 is not in fluid communication with any lumen of catheter tip 26, including lumen 60. Thus, catheter 10 is configured such that fluid withdrawn from lumen 42 via hub 16 may only enter lumen 42 via the respective side opening 14 and fluid may only enter lumen 42 at distal portion 30 of elongated body 12 via the respective side opening 14.

Because catheter tip 26 defines fewer lumens than elongated body 12 (e.g., only one lumen versus two or more defined by elongated body), a total volume of space occupied by catheter tip 26 and a profile of catheter tip 26 may be relatively low without adversely impacting the fluid flow through catheter 10, particularly compared to examples in which catheter tip 26 defines the same number of lumens as elongated body 12 and/or examples in which catheter tip 26 defines two lumens. For example, the outer cross-sectional dimension of the distal-most portion of catheter tip 26 is smaller than the outer cross-sectional dimension of the distal-most portion of elongated body 12 (e.g., at distal end 12B), the cross-sections being taken in a direction orthogonal to longitudinal axis A of catheter 10. In examples in which elongated body 12 is tubular and catheter tip 26 defines a tubular distal-most portion, the cross-sectional dimension may be a diameter. In these examples, because catheter tip 26 distally tapers, as shown in FIG. 11, catheter tip 26 may define a rounded (asymmetric) cone shape or another asymmetric shape (where the line of symmetry is longitudinal axis A of catheter 10).

The distal taper of catheter tip 26 may help minimize the invasiveness of catheter 10, e.g., by reducing adverse interactions with tissue of a patient as catheter 10 is introduced into patient or otherwise navigated through vasculature of a patient. In some examples, at a proximal portion of catheter tip 26, the distal taper is at least partially defined by a compound radius of curvature. For example, at least a proximal portion of catheter tip 26 on the side of catheter tip 26 having the connecting member 70 (described below) can define multiple radii of curvature R_{B}, R_{C}, and R_{D}. In some examples, radius of curvature R_{B} is about 15 mm to about 20 mm, such as about 18 mm, radius of curvature R_{C} is about 1 mm to about 5 mm, such as about 2.55 mm, and radius of curvature R_{D} is about 8 mm to about 15 mm, such as about 11.5 mm.

In other examples, catheter tip 26 may define two or more lumens, and each catheter tip lumen can be in fluid communication with the same or respective lumens 40, 42 of elongated body 12. In some of these examples, catheter tip 26 may have a more symmetrical shape than that shown in FIGS. 10 and 11, such as a substantially symmetrical shape (e.g., symmetrical to the extent permitted by manufacturing tolerances).

In some examples, catheter tip 26 defines an atraumatic distal end 64, which is curved to help reduce any adverse interactions with tissue of a patient as catheter 10 is introduced into patient or otherwise navigated through vasculature of a patient. In some examples, distal end 64 has a radius of curvature RA of about 0.25 mm to about 1 mm, such as about 0.60 mm.

Catheter tip 26 can be mechanically and fluidically connected to elongated body 12 using any suitable technique. In some examples, catheter tip 26 and elongated body 12 are connected via a butt joint, which proximal end 62 of catheter tip is placed directly adjacent to distal end 12B of elongated body 12. In some examples, catheter tip 26 and elongated body 12 are connected via mating parts. For example, as shown in FIGS. 10 and 11, in some examples, catheter tip 26 includes or is otherwise connected to a pair of proximally extending connecting members 68, 70 that are configured to be inserted into lumens 40, 42 of elongated body 12 when assembling (and mechanically connecting) elongated body 12 and catheter tip 26. Thus, in some examples, connecting members 68, 70 may have shapes that correspond to and are configured to be received in and mate with lumen 40, 42. For example, in the example shown in FIGS. 10 and 11, connecting member 68 is configured to be received in lumen 40, and, therefore, has an oval (e.g., a pointed oval or lens) cross-sectional shape, and connecting member 70 is configured to be received in lumen 42 and, therefore, has a kidney cross-sectional shape, the cross-sections being taken in a direction orthogonal to a longitudinal axis of catheter tip 26, which may be parallel to longitudinal axis A of catheter 10 when catheter tip 26 is properly assembled with elongated body 12.

Connecting members 68, 70 are spaced apart from each other to define a space in which septum 44 of elongated body 12 may be received. In examples in which lumen 60 of catheter tip 26 is in fluid communication with lumen 40 of elongated body 12 and lumen 42 of elongated body 12 is not in fluid communication with any lumen of catheter tip 26 when elongated body 12 and catheter tip 26 are properly assembled, connecting member 68 may define a channel 72 configured to fluidically connect lumen 60 of catheter tip 26 with lumen 40 of elongated body 12. In addition, in these examples, connecting member 70 may be configured to fit within lumen 42 of elongated body 12, which may help align catheter tip 26 and elongated body 12 when assembling catheter 10. In some examples, connecting member 70 may be configured to block or otherwise obstruct lumen 42 partially or completely in order to provide a fluid tight seal between catheter tip 26 and elongated body 12 and help prevent fluid from leaking from lumen 42 at distal end 12B of elongated body 12 (e.g., at the interface between catheter tip 26 and elongated body 12).

In examples in which catheter tip 26 defines multiple lumens configured to be in fluid communication with a respective one of lumens 40, 42 of elongated body 12 when elongated body 12 and catheter tip 26 are properly assembled, both connecting members 68, 70 may define a respective channel 72 configured to fluidically connect the lumens of catheter tip 26 with respective lumens 40, 42 of elongated body 12.

In any of the examples described herein, connecting members 68, 70 may engage the elongated body lumens 40, 42 with an interference or frictional fit, forming a substantially fluid tight seal with lumens 40, 42. Alternatively or additionally, connecting members 68, 70 may be secured within with lumens 40, 42 using chemical adhesives or mechanical coupling, such as by welding.

Connecting members 68, 70 may have any suitable length lengths (measured along longitudinal axis A of catheter 10). For example, although connecting member 68 is shown as having a shorter length than connecting member 70, in other examples, connecting member 68 may be longer than connecting member 70. Further, although connecting members 68, 70 having different are shown in FIGS. 10 and 11, in other examples, connecting members 68, 70 may have substantially the same length (e.g., identical lengths, as permitted by manufacturing tolerances).

FIG. 12 is an end view of catheter tip 26 and illustrates an end of catheter tip 26 opposite that shown in FIG. 6. As shown in FIG. 12. For depth perception, the portion of catheter tip 26 that is distal to connecting members 68, 70 is shown with cross-hatching.

While examples catheters are discussed in terms of medical catheters for the administration of fluids and, more particularly, in terms of hemodialysis catheters, the catheters described herein may be used with a range of applications, including other surgical, diagnostic, and related treatments of diseases and body ailments of a subject. Example applications of the catheters described herein include hemodialysis, cardiac, abdominal, urinary, intestinal, in chronic and/or acute applications.

### EXAMPLES

Fluid flow characteristics of a first catheter including two side openings each having a biconcave shape, e.g., as shown in FIGS. 1-5, were compared to a second catheter including two side openings having parallel sides rather than concave sides. The side openings of the first catheter and the side openings of the second catheter had similar lengths and maximum widths. The side openings of the first catheter were partially diametrically opposed (e.g., as shown in FIG. 3A), and the side openings of the second catheter were completely diametrically opposed, thereby defining a symmetrical arrangement of side openings, the line of symmetry being a longitudinal axis of the second catheter. The first and second catheters each defined two D-shaped lumens, one in fluid communication with a respective side opening.

Blood flow through lumens of the first and second catheter (both inflow and outflow directions relative to the side openings) and in or out of the respective side openings were simulated using ANSYS 2019 R2 simulation software, available from Ansys Inc. of Canonsburg, Pa. In particular, the ANSYS CFX computational fluid dynamics tool, the ANSYS SpaceClaim 3D modeling application, and ANSYS Meshing software was used to perform the fluid flow simulations. The average size of the mesh elements was 1 e-4 m.

A steady state analysis was performed to determine the fluid flow parameters of the first and second catheters, except for a residence time distribution (RTD) analysis, described below. The following blood flow regime assumptions were used for the simulations: Non-Newtonian fluid by Carreau-Yasuda viscosity model, incompressible fluid density function of plasma and Hematocrit value (Hct=40%→blood average density=1051 kilograms per cubic meter (kg/m³)), and a relative pressure domain of 4 millimeters of mercury (mmHg), which is a typical average value inside a right atrium during a diastole phase of a cardiac cycle of some patients; buoyancy effect was not taken into consideration considering the multiple positions that can be assumed for the patient during a blood treatment procedure. In addition, an inlet/outlet flow rate through the lumens of the catheters of 200 mL/min was used, an inlet Superior Vena Cava (SVC) flow rate of 2000 mL/min was used, and an outlet SVC opening condition with a relative pressure of 0 Pascals (Pa) was used, For the RTD analysis, a passive tracer with a diffusivity value of 1e-18 m²/s was used.

The outer diameter of the first catheter was 3.9878 mm (a 12 French catheter), and the side openings each had an area of 24.183 mm². The first catheter defined a venous lumen having a cross-sectional area of 3.256 mm² and an arterial lumen having a cross-sectional area of 3.281 mm², the cross-sections being taken in a direction orthogonal to a longitudinal axis of the first catheter. The first catheter included a catheter tip coupled to an elongated body (which defined the venous and arterial lumens), and the catheter tip defined a tip lumen in fluid communication with the arterial lumen and having a cross-sectional area of 0.893 mm² and a diameter of 1.066 mm, the cross-section being taken in a direction orthogonal to a longitudinal axis of the first catheter.

The outer diameter of the second catheter was 3.988 mm (a 12 French catheter), and the side openings each had an area of 20.811 mm². The second catheter defined a venous lumen having a cross-sectional area of 3.211 mm² and an arterial lumen having a cross-sectional area of 3.211 mm², the cross-sections being taken in a direction orthogonal to a longitudinal axis of the second catheter. The second catheter included a catheter tip coupled to an elongated body (which defined the venous and arterial lumens), and the catheter tip defined a tip lumen in fluid communication with the arterial lumen and having a cross-sectional area of 0.894 mm² and a diameter of 1.067 mm, the cross-section being taken in a direction orthogonal to a longitudinal axis of the second catheter.

Fluid flow exiting a respective venous lumen of each catheter and entering an arterial lumen of the respective catheter was simulated using the software described above. The simulation indicated that for the second catheter, a maximum external wall shear stress at the distal ends of the side openings was 48.8 Pa, whereas for the first catheter, a maximum external wall shear stress at the distal ends of the side openings was 38.06 Pa. For another first catheter having a D-shaped lumen and a kidney-shaped lumen (e.g., as shown in FIG. 6), rather than two D-shaped lumens, the maximum external wall shear stress at distal ends of the side openings was 35.70 Pa. The maximum external wall shear stress was the shear stress measured (via simulation) at the external facing wall of the respective catheter. It is believed the biconcave shape of the side openings of the first catheter contributed to the reduction of 22% or 27% of the external wall shear stress relative to the first catheter, which did not include biconcave side openings. As discussed above, reducing shear stress may be desirable because a lower shear stress may result in less hemolysis during dialysis.

The simulation also indicated that for the second catheter, a maximum side wall shear stress near proximal ends of the side openings was 140.34 Pa, whereas for the first catheter, a maximum side wall shear stress near proximal ends of the side openings was 49.32. For another first catheter having a D-shaped lumen and a kidney-shaped lumen, the maximum side wall shear stress near proximal ends of the side openings was 47.20 Pa. The maximum side wall shear stress was the shear stress measured (via simulation) at the side surfaces defining the side openings (e.g., side surfaces corresponding to side surface 48 shown in FIG. 5). It is believed the biconcave shape of the side openings of the first catheter contributed to the reduction of 65% or 68% of the maximum side wall shear stress relative to the first catheter, which did not include biconcave side openings. The simulation also indicates that the biconcave shape of the side openings of the first catheter may result in a reduction of 16% or 18% of the maximum side wall shear stress near the distal ends of the side openings relative to the first catheter.

The simulation indicated that the first catheter showed a slight increase in the maximum internal wall shear stress (about 4% to about 9%) at the proximal ends of the side openings relative to the second catheter. The maximum internal wall shear stress was the shear stress measured (via simulation) at the internal facing wall of the respective catheter.

The residence time distribution was simulated for the first and second catheters in order to determine the impact of the biconcave side openings on blood recirculation. For the residence time distribution simulation, a pulse injection approach was used (an injection time of 0.05 seconds), along with a passive tracer with negligible diffusivity coefficient (1e-18 m²/s) to detect recirculation. A transient analysis was performed for the tracer. It was found that the first catheter including the biconcave side openings reduced the amount of recirculation relative to the recirculation observed with the second catheter. In particular, the percentage of recirculation of the second catheter was determined to be 0.000000043%, whereas for the first catheter, the percentage of recirculation was determined to be 0.00000000016% or 0.00000000031%. The simulation indicates that the first catheter may result in a reduction of 99.62% or 99.28% of recirculation relative to the second catheter.

Example 1: In some examples, a catheter comprises an elongated body defining a body lumen, wherein the elongated body comprises a sidewall defining a side opening in fluid communication with the body lumen, the side opening having a biconcave shape when the elongated body is straight.

Example 2: In some examples of the catheter of example 1, the side opening has a proximal end, a distal end, a first side extending from the proximal end to the distal end, and a second side opposing the first side and extending from the proximal end to the distal end, wherein when the elongated body is straight, the first and second sides curve towards each other between the proximal and distal ends.

Example 3: In some examples of the catheter of example 2, the proximal and distal ends are not aligned along an axis parallel to a longitudinal axis of the elongated body.

Example 4: In some examples of the catheter of example 2 or example 3, the side opening defines a side opening axis intersecting the proximal and distal ends, wherein the first and second sides each defines a curve that curves inwards towards the side opening axis.

Example 5: In some examples of the catheter of example 4, the curve is closest to the side opening axis at a midpoint of the respective first side or second side.

Example 6: In some examples of the catheter of any of examples 1-5, the side opening defines a side opening axis extending between proximal and distal ends of the side opening, and a transverse axis transverse to the side opening axis, and wherein the side opening is symmetric about the transverse axis.

Example 7: In some examples of the catheter of any of examples 1-6, the side opening defines rounded proximal and distal ends.

Example 8: In some examples of the catheter of any of examples 1-7, the lumen is a first body lumen and the side opening is a first side opening, the elongated body further defining a second body lumen, wherein the sidewall of the elongated body defines a second side opening in fluid communication with the second body lumen.

Example 9: In some examples of the catheter of any of examples 1-8, the first and second side openings have the same shape.

Example 10: In some examples of the catheter of any of examples 1-9, the first and second side openings are diametrically opposed.

Example 11: In some examples of the catheter of any of examples 1-10, a proximal end of the first side opening is not aligned with a proximal end of the second side opening along a longitudinal axis of the elongated body.

Example 12: In some examples of the catheter of any of examples 1-11, the catheter further comprises a tapered catheter tip at a distal end of the elongated body, wherein the tapered catheter tip defines a tip lumen in fluid communication with the body lumen.

Example 13: In some examples of the catheter of example 12, the lumen is a first body lumen and the side opening is a first side opening, the elongated body further defining a second body lumen, wherein the sidewall defines a second side opening in fluid communication with the second body lumen, and wherein the tapered catheter tip does not define a lumen in fluid communication with the second body lumen.

Example 14: In some examples of the catheter of example 12 or 13, the tapered catheter tip is asymmetrical about a longitudinal axis of the catheter.

Example 15: In some examples of the catheter of any of examples 12-14, the tapered catheter tip is separate from and secured to a distal portion of the elongated body.

Example 16: In some examples of the catheter of any of examples 12-14, the tapered catheter tip is integrally formed with the elongated body.

Example 17: In some examples, a catheter comprises an elongated body defining a body lumen, wherein the elongated body comprises a sidewall defining a side opening in fluid communication with the body lumen, the side opening having a proximal end, a distal end, a first side extending from the proximal end to the distal end, and a second side opposing the first side and extending from the proximal end to the distal end, wherein when the elongated body is straight, the first and second sides curve towards each other between the proximal and distal ends, and wherein the proximal and distal ends are not aligned along an axis parallel to a longitudinal axis of the elongated body.

Example 18: In some examples of the catheter of example 17, the first and second sides curve towards each other between the proximal and distal ends to define a biconcave shape.

Example 19: In some examples of the catheter of example 17 or 18, the proximal and distal ends of the side opening are not aligned along the axis parallel to the longitudinal axis of the elongated body.

Example 20: In some examples of the catheter of any of examples 17-19, the proximal end of the side opening is circumferentially offset from the distal end of the side opening.

Example 21: In some examples of the catheter of any of examples 17-20, the side opening defines rounded proximal and distal ends.

Example 22: In some examples of the catheter of any of examples 17-21, the lumen is a first body lumen and the side opening is a first side opening, the elongated body further defining a second body lumen, wherein the sidewall defines a second side opening in fluid communication with the second body lumen.

Example 23: In some examples of the catheter of example 22, the first and second side openings have the same shape.

Example 24: In some examples of the catheter of any of example 22 or 22, the first and second side openings are diametrically opposed.

Example 25: In some examples of the catheter of any of examples 22-24, the proximal end of the first side opening is not aligned with the proximal end of the second side opening along the longitudinal axis of the elongated body.

Example 26: In some examples of the catheter of any of examples 17-25, the catheter further comprises a tapered catheter tip at a distal end of the elongated body, wherein the tapered catheter tip defines a tip lumen in fluid communication with the body lumen.

Example 27: In some examples of the catheter of example 26, the lumen is a first body lumen and the side opening is a first side opening, the elongated body further defining a second body lumen, wherein the sidewall defines a second side opening in fluid communication with the second body lumen, and wherein the tapered catheter tip does not define a lumen in fluid communication with the second body lumen.

Example 28: In some examples of the catheter of example 26 or 27, the tapered catheter tip is asymmetrical about the longitudinal axis of the catheter.

Example 29: In some examples, a catheter comprises an elongated body defining a body lumen, wherein the elongated body comprises a sidewall defining a side opening in fluid communication with the body lumen, the side opening having a proximal end, a distal end, a first side extending from the proximal end to the distal end, and a second side opposing the first side and extending from the proximal end to the distal end, wherein each of the first side and the second side defines a continuous curve that curves towards the other of the first side or the second side in a direction towards a midpoint of the respective first or second side.

Example 30: In some examples of the catheter of example 29, the first and second sides curve towards each other between the proximal and distal ends of the side opening to define a biconcave shape.

Example 31: In some examples of the catheter of example 29 or 30, the proximal and distal ends of the side opening are not aligned along an axis parallel to a longitudinal axis of the elongated body.

Example 32: In some examples of the catheter of any of examples 29-31, the proximal end of the side opening is circumferentially offset from the distal end of the side opening.

Example 33: In some examples of the catheter of any of examples 29-32, the side opening defines rounded proximal and distal ends.

Example 34: In some examples of the catheter of any of examples 29-33, the lumen is a first body lumen and the side opening is a first side opening, the elongated body further defining a second body lumen, wherein the sidewall defines a second side opening in fluid communication with the second body lumen.

Example 35: In some examples of the catheter of example 34, the first and second side openings have the same shape.

Example 36: In some examples of the catheter of example 34 or 35, the first and second side openings are diametrically opposed.

Example 37: In some examples of the catheter of any of examples 34-36, the proximal end of the first side opening is not aligned with the proximal end of the second side opening along a longitudinal axis of the elongated body.

Example 38: In some examples of the catheter of any of examples 29-37, the catheter further comprises a tapered catheter tip at a distal end of the elongated body, wherein the tapered catheter tip defines a tip lumen in fluid communication with the body lumen.

Example 39: In some examples of the catheter of example 38, the lumen is a first body lumen and the side opening is a first side opening, the elongated body further defining a second body lumen, wherein the sidewall defines a second side opening in fluid communication with the second body lumen, and wherein the tapered catheter tip does not define a lumen in fluid communication with the second body lumen.

Example 40: In some examples of the catheter of example 38 or example 39, the tapered catheter tip is asymmetrical about a longitudinal axis of the catheter.

Example 41: A method of using any of the catheters of examples 1-40.

Example 42: A method of making any of the catheters of examples 1-40.

Various examples have been described. These and other examples are within the scope of the following claims.

1: A catheter comprising:
an elongated body defining a body lumen, wherein the elongated body comprises a sidewall defining a side opening in fluid communication with the body lumen, the side opening having a biconcave shape when the elongated body is straight.

2: The catheter of claim 1, wherein the side opening has a proximal end, a distal end, a first side extending from the proximal end to the distal end, and a second side opposing the first side and extending from the proximal end to the distal end, wherein when the elongated body is straight, the first and second sides curve towards each other between the proximal and distal ends.

3: The catheter of claim 2, wherein the proximal and distal ends are not aligned along an axis parallel to a longitudinal axis of the elongated body.

4: The catheter of claim 2, wherein the side opening defines a side opening axis intersecting the proximal and distal ends, wherein the first and second sides each defines a curve that curves inwards towards the side opening axis.

5: The catheter of claim 4, wherein the curve is closest to the side opening axis at a midpoint of the respective first side or second side.

6: The catheter of claim 1, wherein the side opening defines a side opening axis extending between proximal and distal ends of the side opening, and a transverse axis transverse to the side opening axis, and wherein the side opening is symmetric about the transverse axis.

7: The catheter of claim 1, wherein the side opening defines rounded proximal and distal ends.

8: The catheter of claim 1, wherein the lumen is a first body lumen and the side opening is a first side opening, the elongated body further defining a second body lumen, wherein the sidewall of the elongated body defines a second side opening in fluid communication with the second body lumen.

9: (canceled)

10: The catheter of claim 8, wherein the first and second side openings are diametrically opposed.

11: The catheter of claim 8, wherein a proximal end of the first side opening is not aligned with a proximal end of the second side opening along a longitudinal axis of the elongated body.

12: The catheter of claim 1, further comprising a tapered catheter tip at a distal end of the elongated body, wherein the tapered catheter tip defines a tip lumen in fluid communication with the body lumen.

13: The catheter of claim 12, wherein the lumen is a first body lumen and the side opening is a first side opening, the elongated body further defining a second body lumen, wherein the sidewall defines a second side opening in fluid communication with the second body lumen, and wherein the tapered catheter tip does not define a lumen in fluid communication with the second body lumen.

14-16. (canceled)

17: A catheter comprising:
an elongated body defining a body lumen, wherein the elongated body comprises a sidewall defining a side opening in fluid communication with the body lumen, the side opening having a proximal end, a distal end, a first side extending from the proximal end to the distal end, and a second side opposing the first side and extending from the proximal end to the distal end, wherein when the elongated body is straight, the first and second sides curve towards each other between the proximal and distal ends, and wherein the proximal and distal ends are not aligned along an axis parallel to a longitudinal axis of the elongated body.

18: The catheter of claim 17, wherein the first and second sides curve towards each other between the proximal and distal ends to define a biconcave shape.

19: The catheter of claim 17, wherein the proximal and distal ends of the side opening are not aligned along the axis parallel to the longitudinal axis of the elongated body.

20: The catheter of claim 17, wherein the proximal end of the side opening is circumferentially offset from the distal end of the side opening.

21: (canceled)

22: The catheter of claim 17, wherein the lumen is a first body lumen and the side opening is a first side opening, the elongated body further defining a second body lumen, wherein the sidewall defines a second side opening in fluid communication with the second body lumen, wherein the first and second side openings have the same shape.

23-25. (canceled)

26: The catheter of claim 17, further comprising a tapered catheter tip at a distal end of the elongated body, wherein the tapered catheter tip defines a tip lumen in fluid communication with the body lumen, wherein the tapered catheter tip is asymmetrical about the longitudinal axis of the catheter.

27-28. (canceled)

29: A catheter comprising:
an elongated body defining a body lumen, wherein the elongated body comprises a sidewall defining a side opening in fluid communication with the body lumen, the side opening having a proximal end, a distal end, a first side extending from the proximal end to the distal end, and a second side opposing the first side and extending from the proximal end to the distal end, wherein each of the first side and the second side defines a continuous curve that curves towards the other of the first side or the second side in a direction towards a midpoint of the respective first or second side.

30: The catheter of claim 29, wherein the first and second sides curve towards each other between the proximal and distal ends of the side opening to define a biconcave shape.

31: The catheter of claim 29, wherein the proximal and distal ends of the side opening are not aligned along an axis parallel to a longitudinal axis of the elongated body.

32: The catheter of claim 29, wherein the proximal end of the side opening is circumferentially offset from the distal end of the side opening.

33. (canceled)

34: The catheter of claim 29, wherein the lumen is a first body lumen and the side opening is a first side opening, the elongated body further defining a second body lumen, wherein the sidewall defines a second side opening in fluid communication with the second body lumen, wherein the first and second side openings have the same shape.

35-40. (canceled)
